(19) United States
(12) Patent Application Publication
(10) Pub. No.: US 2024/0426721 A1
(43) Pub. Date: Dec. 26, 2024

(54) SALIVA PRE-PROCESSING DEVICE AND PRE-PROCESSING METHOD

(71) Applicant: IUCF-HYU (INDUSTRY-UNIVERSITY COOPERATION FOUNDATION HANYANG UNIVERSITY), Seoul (KR)

(72) Inventors: Kyung Gyun HWANG, Seoul (KR); Hyun Ju YU, Seoul (KR); Eun Ju JEONG, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 18/700,863

(22) PCT Filed: Oct. 12, 2022
(86) PCT No.: PCT/KR2022/015418

§ 371 Date: Apr. 12, 2024

(30) Foreign Application Priority Data
Oct. 13, 2021 (KR) 10-2021-0135412

(51) Int. Cl. G01N 1/40; B01D 29/01; B01D 29/05; B01D 29/35; B01D 29/56; G01N 33/487

(52) U.S. Cl. CPC G01N 1/4077; B01D 29/014; B01D 29/05; B01D 29/35; B01D 29/56; G01N 33/487; B01D 2201/02; B01D 2201/0415; B01D 2201/184; G01N 2001/4088

(57) ABSTRACT

A saliva pre-processing device is provided. The saliva pre-processing device comprises: a first filter module for filtering out impurities in saliva such that the impurities in the saliva remain on a filter; and a second filter module which receives the saliva from which the impurities are filtered out from the first filter module, and concentrates a target material in the saliva such that the target material in the saliva remains on a filter, wherein the first and second filter modules may have a porous structure.

### DESCRIPTION

### Technical Field

The present invention relates to a device and a method for pre-processing saliva, and more specifically, to a device and a method for pre-processing saliva, which concentrate a target material in the saliva to diagnose the target material in vitro.

### Background Art

Saliva, which may be considered a representative oral sample, is responsible for functions necessary for oral health such as digestion, immunity, lubrication, cushioning, cleaning, taste, and healing in an oral cavity, and is a sample containing the most direct and diverse information about bacterial environment in the oral cavity. The saliva is mostly composed of water, and only about 2% thereof is a sample mixed with various components such as electrolytes, digestive enzymes, food residues, bacteria and metabolites, oral epithelium, immunoglobulins, and proteins.

The saliva contains various biomolecules that reflect health conditions of a human body, such as bacteria, proteins, nucleic acids, etc., and may be easily collected by a non-invasive method, but since the samples may be deteriorated and collected saliva contaminated, a process cumbersome of removing impurities other than a target material is inevitably accompanied to high obtain reliability results. Therefore, in order to accurately analyze the saliva, saliva collection methods according to various parts and conditions need to be introduced, and research on pre-processing methods suitable for various collected saliva is required.

### DISCLOSURE

### Technical Problem

One technical problem to be solved by the present invention is to provide a device and a method for pre-processing saliva in which a disease diagnosis process through the saliva is simplified.

Another technical problem to be solved by the present invention is to provide a device and a method for pre-processing saliva, which facilitate removal of impurities in the saliva.

Still another technical problem to be solved by the present invention is to provide a device and a method for pre-processing saliva, which facilitate concentration of a target material in the saliva.

Still another technical problem to be solved by the present invention is to provide a device and a method for pre-processing saliva, which may purify impurities in the saliva and concentrate a target material in the saliva through one device.

The technical problems to be solved by the present invention are not limited to those described above.

### Technical Solution

To solve the above technical problems, the present invention provides a saliva pre-processing device.

According to one embodiment, the saliva pre-processing device may include: a first filter module configured to purify impurities in saliva such that the impurities in the saliva remain on a filter; and a second filter module configured to receive the saliva from which the impurities are purified from the first filter module to concentrate a target material in the saliva such that the target material in the saliva remains on the filter, in which the first and second filter modules have a porous structure, and a pore size of the second filter module is smaller than a pore size of the first filter module.

According to one embodiment, the first filter module may include: a first filter body having an inlet and an outlet disposed to face each other; and a first filter disposed between the inlet and the outlet in the first filter body, and the first filter may have a shape that is gradually convex from an outside toward a center thereof, and a convex portion of the first filter may be disposed to face the inlet of the first filter body.

According to one embodiment, the first filter module may further include a plurality of supporters having a rod shape extending from the outside toward the center of the first filter, and configured to support the first filter while being disposed below the first filter, and the supporters may have a slope formed in a direction from one end to the other end thereof, so that the convex portion of the first filter is formed by the supporters.

According to one embodiment, the second filter module may include: a second filter body having an inlet and an outlet disposed to face each other, and having a conical shape with a diameter that gradually decreases from an upper portion in which the inlet is formed to a lower portion in which the outlet is formed; and a second filter having a rod shape extending in one direction and disposed on an inner surface of the second filter body.

According to one embodiment, the saliva introduced from the first filter module to the second filter module may move along an inner surface of the second filter module so that the target material is concentrated by the second filter, and the saliva in which the target material is concentrated through the second filter may be discharged out of the second filter module.

According to one embodiment, a plurality of second filters may be disposed on the inner surface of the filter body, and any one of the plurality of second filters may include a pH indicator indicating a pH of the saliva.

According to one embodiment, the second filter may be replaced depending on a type of the target material, and a pore size of the second filter may vary depending on the type of the target material.

According to one embodiment, the first filter module and the second filter module may include any one of mixed cellulose ester (MCE), polyethersulfone (PES), cellulose acetate (CA), Nylon, Teflon, polyvinylidene fluoride (PVDF), and polytetrafluoroethylene (PTFE).

According to one embodiment, the saliva pre-processing device may further include: a saliva cartridge coupled to the first filter module, and configured to store the saliva supplied to the first filter module; and a saliva injection module coupled to the saliva cartridge, and configured to apply a pressure to the saliva cartridge such that the saliva is supplied from the saliva cartridge to the first filter module.

According to one embodiment, the saliva cartridge may include a rubber packing that makes contact with the saliva injection module and forms a space for storing the saliva, and a pH indicator disposed in the space for storing the saliva, and as the saliva injection module applies the pressure to the rubber packing, the saliva stored in the saliva storage space may be supplied to the first filter module.

To solve the above technical problems, the present invention provides a saliva pre-processing method.

According to one embodiment, the saliva pre-processing method may include: preparing a saliva cartridge in which saliva is stored; supplying the saliva stored in the saliva cartridge to a first filter having a pore of a first size; purifying impurities in the saliva such that the impurities in a material of the saliva remain on the first filter, and passing the saliva from which the impurities are purified through the first filter; supplying the saliva from which the impurities are purified to a second filter formed with a pore of a second size that is smaller than the first size; and concentrating a target material in the saliva such that the target material in the saliva remains on the second filter, and passing the saliva in which the target material is concentrated through the second filter.

According to one embodiment, the target material may include any one of protein, metabolite, and exosome.

### Advantageous Effects

The saliva pre-processing device according to the embodiment of the present invention may include: a first filter module configured to purify impurities in saliva such that the impurities in the saliva remain on a filter; and a second filter module configured to receive the saliva from which the impurities are purified from the first filter module to concentrate a target material in the saliva such that the target material in the saliva remains on the filter, in which the first and second filter modules have a porous structure, and a pore size of the second filter module is smaller than a pore size of the first filter module.

Accordingly, since a purification process of removing the impurities in the saliva and a process of concentrating the target material in the saliva may be all performed through one device, in vitro diagnosis using the saliva may be easily performed.

### DESCRIPTION OF DRAWINGS

FIG. 1 is a perspective view of a saliva pre-processing device according to an embodiment of the present invention.

FIG. 2 is a schematic sectional view of the saliva pre-processing device according to the embodiment of the present invention.

FIG. 3 is a perspective view of a first filter module included in the saliva pre-processing device according to the embodiment of the present invention.

FIG. 4 is a cut-out perspective view of the first filter module included in the saliva pre-processing device according to the embodiment of the present invention.

FIG. 5 is a sectional view of the first filter module included in the saliva pre-processing device according to the embodiment of the present invention.

FIG. 6 is a perspective view of a second filter module included in the saliva pre-processing device according to the embodiment of the present invention.

FIG. 7 is a cut-out perspective view of the second filter module included in the saliva pre-processing device according to the embodiment of the present invention.

FIG. 8 is a sectional view of the second filter module included in the saliva pre-processing device according to the embodiment of the present invention.

FIG. 9 is a perspective view of a saliva cartridge included in the saliva pre-processing device according to the embodiment of the present invention.

FIG. 10 is a schematic sectional view of the saliva cartridge included in the saliva pre-processing device according to the embodiment of the present invention.

FIG. 11 is a perspective view of a saliva injection module included in the saliva pre-processing device according to the embodiment of the present invention.

FIG. 12 is a view for explaining discharge of the saliva in the saliva cartridge through the saliva injection module included in the saliva pre-processing device according to the embodiment of the present invention.

FIGS. 13 and 14 are views explaining operations of the saliva pre-processing device according to the embodiment of the present invention.

FIG. 15 is a graph for confirming size distribution of biomarkers used in the saliva pre-processing device according to the embodiment of the present invention.

FIG. 16 is a graph showing results of filtering the biomarkers separated in FIG. 15 by the saliva pre-processing device according to the present invention.

FIG. 17 is a photograph obtained by capturing a state in which the biomarkers are filtered through the saliva pre-processing device according to the embodiment of the present invention.

### MODE FOR INVENTION

Hereinafter, preferred embodiments of the present invention will be described in detail with reference to the accompanying However, drawings. the present invention may be embodied in different forms and should not be construed as limited to the embodiments set forth herein. Rather, the embodiments are provided so that this disclosure will be thorough and complete, and will fully convey the scope of the present invention to those skilled in the art.

In the present specification, it will be understood that when an element is referred to as being “on” another element, it can be formed directly on the other element or intervening elements may be present. In the drawings, the thicknesses of layers and regions are exaggerated for clarity.

In addition, it will be also understood that although the terms first, second, third, etc. may be used herein to describe various elements, these elements should not be limited by these terms. These terms are only used to distinguish one element from another element. Thus, a first element in some embodiments may be termed a second element in other embodiments without departing from the teachings of the present invention. Embodiments explained and illustrated herein include their complementary counterparts. As used herein, the term “and/or” includes any and all combinations of one or more of the associated listed elements.

The singular expression also includes the plural meaning as long as it does not differently mean in the context. In addition, the terms “comprise”, “have” etc., of the description are used to indicate that there are features, numbers, steps, elements, or combinations thereof, and they should not exclude the possibilities of combination or addition of one or more features, numbers, operations, elements, or a combination thereof. Furthermore, it will be understood that when an element is referred to as being “connected” or “coupled” to another element, it may be directly connected or coupled to the other element or intervening elements may be present.

In addition, when detailed descriptions of related known functions or constitutions are considered to unnecessarily cloud the gist of the present invention in describing the present invention below, the detailed descriptions will not be included.

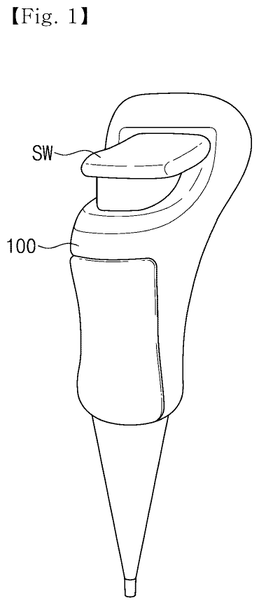

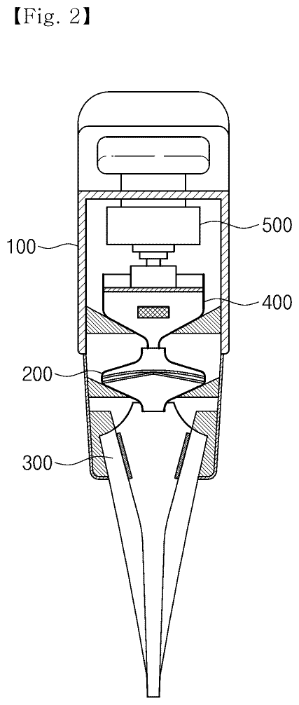

FIG. 1 is a perspective view of a saliva pre-processing device according to an embodiment of the present invention, and FIG. 2 is a schematic sectional view of the saliva pre-processing device according to the embodiment of the present invention.

Referring to FIGS. 1 and 2, the saliva pre-processing device according to the embodiment of the present invention may include a housing 100, a first filter module 200, a second filter module 300, a saliva cartridge 400, and a saliva injection module 500. Hereinafter, each configuration will be described.

The housing 100 may form an appearance of the saliva pre-processing device. In addition, the housing 100 may be provided so as to support and accommodate various components installed therein. According to the embodiment, the first filter module 200, the second filter module 300, the saliva cartridge 400, and the saliva injection module 500 may be disposed in the housing 100, and the first filter module 200, the second filter module 300, the saliva cartridge 400, and the saliva injection module 500 may be supported and accommodated by the housing 100.

A switch SW for controlling the saliva injection module 500 may be provided on an upper end of the housing 100. The switch SW may be formed through the inside and outside of the housing 100, and the saliva injection module 500 may be controlled from the outside of the housing 100 through the switch SW. An operation of the saliva injection module 500 through the switch SW will be described later.

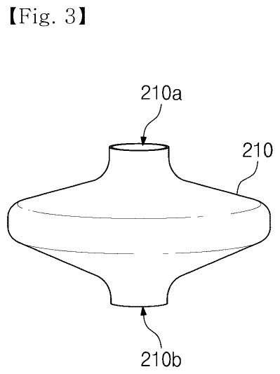

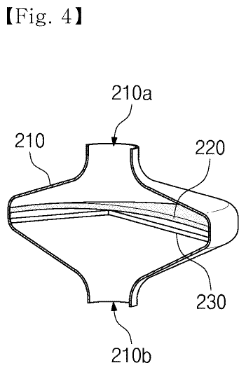

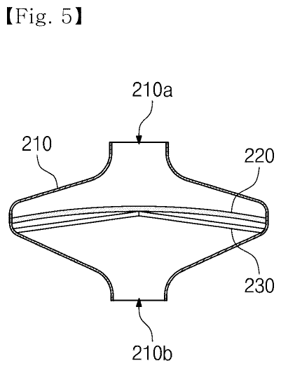

FIG. 3 is a perspective view of a first filter module included in the saliva pre-processing device according to the embodiment of the present invention, FIG. 4 is a cut-out perspective view of the first filter module included in the saliva pre-processing device according to the embodiment of the present invention, and FIG. 5 is a sectional view of the first filter module included in the saliva pre-processing device according to the embodiment of the present invention.

Referring to FIGS. 4 and 5, the first filter module 200 may include a first filter body 210, a first filter 220, and a supporter 230.

The first filter body 210 may form an appearance of the first filter module 200. According to one embodiment, the first filter body 210 may have a first inlet 210a and a first outlet 210b disposed to face each other, and may have a shape with a diameter that gradually increases from the first inlet 210a or the first outlet 210b to a portion between the first inlet 210a and the first outlet 210b. In other words, the diameter gradually increases and gradually decreases again from the first inlet 210a to the first outlet 210b. For example, the first filter body 210 may have a spinning top shape as shown in FIGS. 3 to 5.

The first filter 220 is disposed inside the first filter body 210, and may be disposed between the first inlet 210a and the first outlet 210b. According to one embodiment, the first filter 220 may be provided in a region having the largest diameter of the first filter body 210. The first filter 220 may purify impurities (e.g., food residues, etc.) in the saliva introduced through the first inlet 210a. More specifically, the first filter 220 may purify the impurities in the saliva such that the impurities in the saliva remain on the first filter 220. That is, when the saliva is filtered through the first filter 220, the impurities in the saliva may remain on the first filter 220, and the saliva from which the impurities are removed may be discharged through the first outlet 210b while passing through the first filter 220.

The first filter 220 may have a porous structure. For example, the first filter 220 may have a pore size of about 1,000 nm. In addition, the first filter 220 may include any one of mixed cellulose ester (MCE), polyethersulfone (PES), cellulose acetate (CA), Nylon, Teflon, polyvinylidene fluoride (PVDF), and polytetrafluoroethylene (PTFE).

According to one embodiment, the first filter 220 may have a shape that is gradually convex from the outside to the center of the first filter 220. Further, a convex portion of the first filter 220 may be disposed to face the first inlet 210a of the first filter body 210. Accordingly, when the saliva introduced through the first inlet 210a is provided on the first filter 220, the saliva may flow from the center of the first filter 220 to the outside of the first filter 220. That is, the saliva provided on the first filter 220 may be uniformly spread and diffused on a surface of the first filter 220. Therefore, the saliva may be purified using the entire parts of the first filter 220, so that purification efficiency and speed of the impurities in the saliva may be improved.

The supporter 230 is disposed inside the first filter body 210, and may be disposed between the first filter 220 and the first outlet 210b. That is, the supporter 230 may be disposed below the first filter 220.

According to one embodiment, a plurality of supporters 230 are disposed below the first filter 220, so that the first filter 220 may be supported by the plurality of supporters 230. Accordingly, despite a pressure caused by the saliva injection module 400 to be described later, a position of the first filter 220 may be fixed, the first filter 220 may stably perform filtering even in a high-pressure environment by the saliva injection module 400, and as a result, the purification of impurities from the saliva may be easily performed.

According to one embodiment, the supporters 230 may have a rod shape extending in a direction from the outside to the center of the first filter 220. In addition, the supporters 230 may have a slope formed in a direction from one end to the other end thereof. Therefore, the convex portion of the first filter 220 may be formed by the plurality of supporters 230 disposed below the first filter 220.

It is obvious to those skilled in the art that the specific form, shape, and number of the supporters 230 are not limited to FIGS. 3 to 5, and may be variously modified. According to the technical idea of the present application, the supporter 230 may be variously modified within the scope of implementing a function of stably supporting the first filter 220 and supporting the first filter 220 such that the first filter 220 may have a convex shape toward the first inlet 210a.

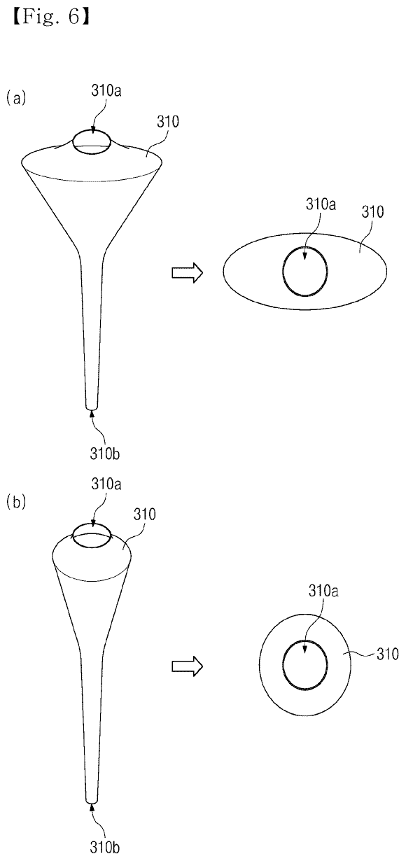

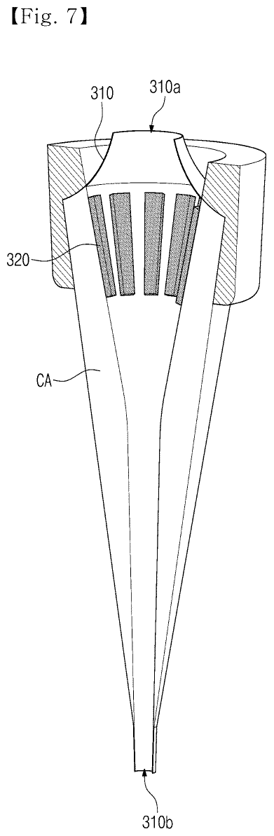

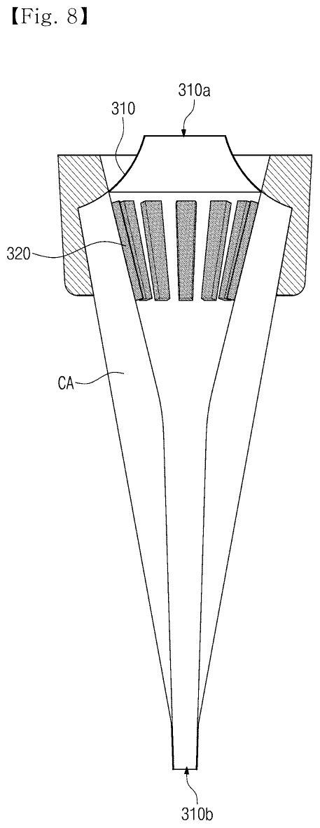

FIG. 6 is a perspective view of a second filter module included in the saliva pre-processing device according to the embodiment of the present invention, FIG. 7 is a cut-out perspective view of the second filter module included in the saliva pre-processing device according to the embodiment of the present invention, and FIG. 8 is a sectional view of the second filter module included in the saliva pre-processing device according to the embodiment of the present invention.

Referring to FIGS. 6 to 8, the second filter module 300 may include a second filter body 310 and a second filter 320.

The second filter body 310 may form an appearance of the second filter module 300. According to one embodiment, the second filter body 310 may have a second inlet 310a and a second outlet 310b disposed to face each other, and may have a conical shape with a diameter that gradually decreases from an upper portion in which the second inlet 310a is formed to a lower portion in which the second outlet is formed 310b. For example, an upper portion of the second filter body 310 may have an elliptical shape as shown in FIG. 6(a), or the upper portion of the second filter body 310 may have a circular shape as shown in FIG. 6(b).

According to one embodiment, the second filter body 310 may include an inner body and an outer body, and a collection space CA may be formed between the inner body and the outer body. Moreover, according to one embodiment, an end of the second filter body 310 may have a small hole or may be blocked.

The second filter module 300 may be coupled to the first filter module 200. Specifically, the first filter body 210 and the second filter body 310 may be coupled such that the first outlet 210b of the first filter module 200 communicates with the second inlet 310a of the second filter module 300. Accordingly, the saliva discharged from the first outlet 210b of the first filter module 200 may flow into the second filter module 300 through the second inlet 310a of the second filter module 300.

The second filter 320 may be disposed inside the second filter body 310, and may be disposed between the second inlet 310a and the second outlet 310b. In addition, a plurality of second filters 320 may be disposed on an inner surface of the second filter body 310. According to one embodiment, the second filter 320 may have a sheet shape extending in one direction. It is obvious to those skilled in the art that the shape of the second filter 320 may be variously changed.

The saliva introduced from the first filter module 200 to the second filter module 300 may move along the inner surface of the second filter body 310, and may be provided to the second filter 320. According to one embodiment, a region of the inner surface of the second filter body 310 from the second inlet 310a to the second filter 320 may have hydrophobic properties. Accordingly, the saliva introduced to the second filter module 300 may easily move to the second filter 320 along the inner surface of the second filter body 310.

According to one embodiment, the second filter 320 may also have a porous structure, and a pore size of the second filter 320 may be smaller than a pore size of the first filter 310. For example, the second filter 320 may include any one of mixed cellulose ester (MCE), polyethersulfone (PES), cellulose acetate (CA), Nylon, Teflon, polyvinylidene fluoride (PVDF), and polytetrafluoroethylene (PTFE).

Therefore, the second filter 320 may concentrate a target material in the saliva by receiving the saliva from which the impurities are purified from the first filter module 200. For example, the target material may include any one of protein, metabolite, and exosome.

More specifically, the second filter 320 may concentrate the target material in the saliva such that the target material in the saliva remains on the second filter 320. That is, when the saliva is filtered through the second filter 320, the target material may remain on the second filter 320. The saliva from which the target material is removed may be discharged out of the second filter module 300 after moving to the collection space CA.

According to one embodiment, the second filter 320 may be replaced depending on a type of the target material. Further, the pore size of the second filter 320 to be replaced may vary depending on the type of the target material. For example, when the target material is protein, the pore size of the second filter 320 may be about 3 kDA. In contrast, when the target material is metabolite, the pore size of the second filter 320 may be 10 Da to 100 Da. In contrast, when the target material is exosome, the pore size of the second filter 320 may be 20 nm to 30 nm.

According to one embodiment, any one of the plurality of second filters 320 disposed on the inner surface of the second filter body 310 may include an indicator indicating a state of the saliva. For example, any one of the plurality of second filter 320 may include an indicator indicating a pH of the saliva.

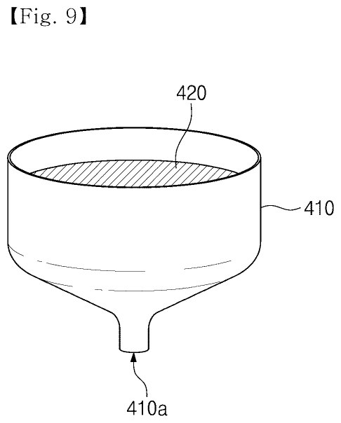

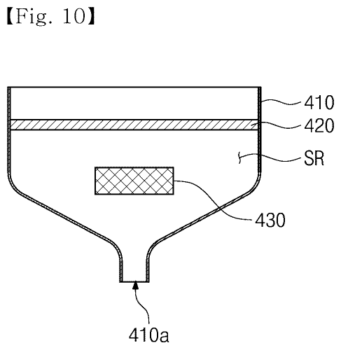

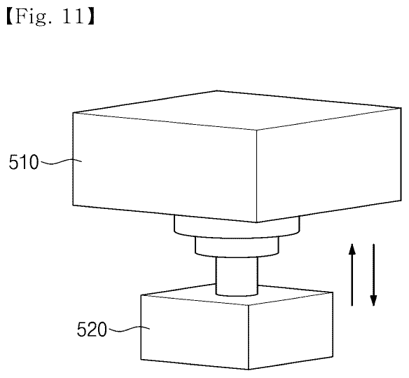

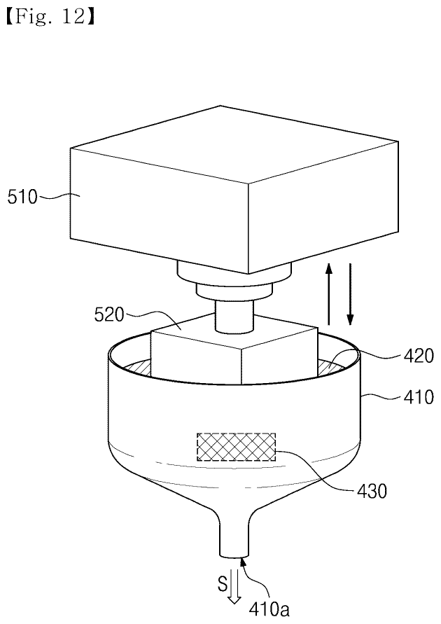

FIG. 9 is a perspective view of a saliva cartridge included in the saliva pre-processing device according to the embodiment of the present invention, FIG. 10 is a schematic sectional view of the saliva cartridge included in the saliva pre-processing device according to the embodiment of the present invention, FIG. 11 is a perspective view of a saliva injection module included in the saliva pre-processing device according to the embodiment of the present invention, and FIG. 12 is a view for explaining discharge of the saliva in the saliva cartridge through the saliva injection module included in the saliva pre-processing device according to the embodiment of the present invention.

Referring to FIGS. 9 and 10, the saliva cartridge 400 may include a cartridge body 410, a rubber packing 420, and a pH indicator 430.

The cartridge body 410 may form an appearance of the saliva cartridge 400. According to one embodiment, as formed in FIGS. 9 and 10, the cartridge body 410 may have a funnel shape.

The rubber packing 420 may be disposed inside the cartridge body 410 to form a saliva space SR for storing the saliva. According to one embodiment, the cartridge body 410 may have a saliva outlet 410a, in which the saliva outlet 410a may communicate with the saliva space SR. Further, in the saliva space SR, not only the saliva may be stored, but also the pH indicator may be disposed. As the pH indicator 430 is disposed in the saliva space SR, the state of the saliva may be confirmed before determining the saliva through the first filter module 200.

The saliva cartridge 400 may be coupled to the first filter module 200. Specifically, the cartridge body 410 and the first filter body 210 may be coupled such that the saliva outlet 410a of the saliva cartridge 400 communicates with the first inlet 210a of the first filter module 200. Accordingly, the saliva discharged from the saliva space SR through the saliva outlet 410a may be provided from the saliva cartridge 400 to the first filter module 200.

Referring to FIGS. 11 and 12, the saliva injection module 500 may include a motor pump 510 and a connection member 520. According to one embodiment, the connection member 520 may be coupled to the rubber packing 420 of the saliva cartridge 400 so that a pressure generated in the motor pump 510 is transmitted to an inner space of the saliva cartridge 400.

As described above, the motor pump 510 may apply the pressure to the inner space of the saliva cartridge 400 through the connection member 520 and the rubber packing 420 in a state where the connection member 520 is coupled to the rubber packing 420. Therefore, saliva S stored in the saliva space SR may be discharged from the saliva cartridge 400 to move to the first filter module 200. According to one embodiment, the motor pump 510 may be controlled through the switch SW of the housing 100.

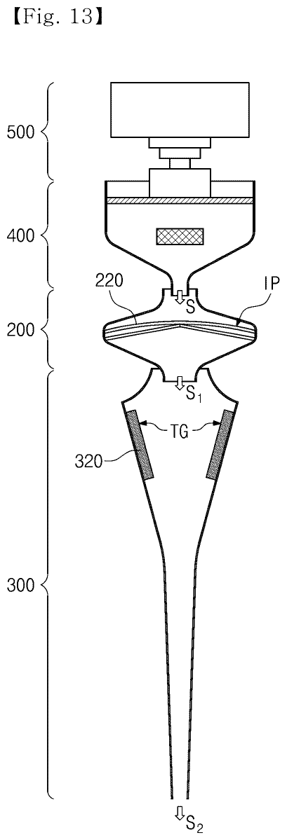

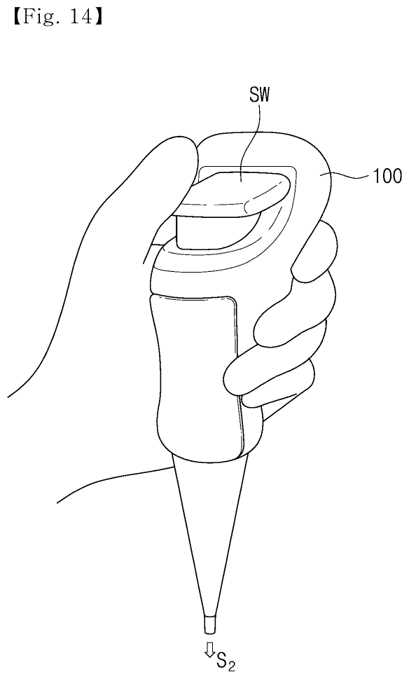

FIGS. 13 and 14 are views explaining operations of the saliva pre-processing device according to the embodiment of the present invention.

Referring to FIGS. 13 and 14, the pressure may be applied to the saliva cartridge 400 through the saliva injection module 500 in a state where the saliva S is stored in the saliva space SR of the saliva cartridge 400. Accordingly, the saliva S may be provided from the saliva cartridge 400 to the first filter module 200.

The saliva S provided to the first filter module 200 may be purified through the first filter 220. Specifically, impurities IP in the saliva S may remain on the first filter 220, and the saliva S from which the impurities IP are removed may pass through the first filter 220. Saliva S₁ from which the impurities IP are removed through the first filter 220 may be provided from the first filter module 200 to the second filter module 300.

The saliva S₁ provided to the second filter module 300 may have a target material TG concentrated through the second filter 320. Specifically, the target material TG in the saliva S₁ may remain on the first filter 320, and saliva S₂ from which the target material TG is removed may pass through the first filter 200. The saliva S₂ in which the target material TG is concentrated through the second filter 320 may be discharged out of the second filter module 300.

The target material TG remaining on the second filter 320 may be mixed with a buffer solution and provided to a disease diagnosis kit, and various diseases may be diagnosed through the disease diagnosis kit. Specifically, when protein, metabolite, and exosome are concentrated as the target material TG, salivary gland function disease, rheumatoid disease, dry mouth, periodontal disease, oral cancer, and other cancers may be diagnosed through the disease diagnosis kit.

That is, in the saliva pre-processing device according to the embodiment of the present invention, both a purification process of removing the impurities in the saliva S and a process of concentrating the target material in the saliva S may be performed through one device. Accordingly, in vitro diagnosis using saliva may be easily performed.

Further, in the saliva pre-processing device according to the embodiment of the present invention, the first filter module 200, the second filter module 300, the saliva cartridge 400, and the saliva injection module 500 may be separately prepared, and the respective components may be coupled to and separated from each other. Accordingly, not only the first and second filters 220 and 320 disposed inside the first and second filter modules 200 and 300 may be easily replaced, but also maintenance of the saliva cartridge 400 and the saliva injection module 500 may be easily performed. In addition, according to one modified example, the housing 100 of the saliva pre-processing device may be divided into an upper housing in which the saliva cartridge 400 and the saliva injection module 500 are accommodated, and a lower housing in which the first filter module 200 and the second filter module 300 are accommodated. The upper housing and the lower housing may be individually prepared, and may be coupled to and separated from each other.

Hereinabove, the saliva pre-processing device according to the embodiment of the present invention has been described. Hereinafter, a saliva pre-processing method according to an embodiment of the present invention will be described.

The saliva pre-processing method according to the embodiment of the present invention may include: a step S100 of preparing a saliva cartridge in which saliva is stored; a step S200 of supplying the saliva stored in the saliva cartridge to a first filter; a step S300 of purifying impurities in the saliva through the first filter; a step S400 of supplying the saliva from which the impurities are purified to a second filter; and a step S500 of concentrating a target material in the saliva through the second filter. Hereinafter, each step will be described.

In step S100, a saliva cartridge in which the saliva is stored may be prepared. According to one embodiment, various indicators capable of confirming a state of the saliva may be disposed inside the saliva cartridge. For example, a pH indicators capable of confirming a pH of the saliva may be disposed in the saliva cartridge. Therefore, it is possible to confirm the state of the saliva stored in the saliva cartridge before filtering the saliva through the first filter and the second filter.

In step S200, the saliva stored in the saliva cartridge may be supplied to the first filter. According to one embodiment, the first filter may have a porous structure having a pore of a first size.

In step S300, impurities (e.g., food residues etc.) in the saliva may be purified through the first filter. Specifically, the impurities in the saliva may be purified such that the impurities in the saliva remain on the first filter, and the saliva from which the impurities are purified may pass through the first filter.

In step S400, the saliva stored in the saliva cartridge may be supplied to the second filter. According to one embodiment, the second filter may have a porous structure having a pore of a second size that is smaller than the first size.

In step S500, a target material (e.g., protein, metabolite, exosome, etc.) in the saliva may be purified through the second filter. Specifically, the target material in the saliva may be concentrated such that the target material in the saliva remains on the second filter, and the saliva in which the target material is concentrated may pass through the second filter.

Thereafter, the target material remaining on the second filter may be mixed with a buffer solution, and then provided to a disease diagnosis kit. Therefore, in vitro diagnosis of the saliva may be performed through the disease diagnosis kit.

Hereinabove, the pre-processing method saliva according to the embodiment of the present invention has been described. Hereinafter, specific experimental examples and characteristic evaluation results of the saliva pre-processing device according to the embodiment of the present invention will be described.

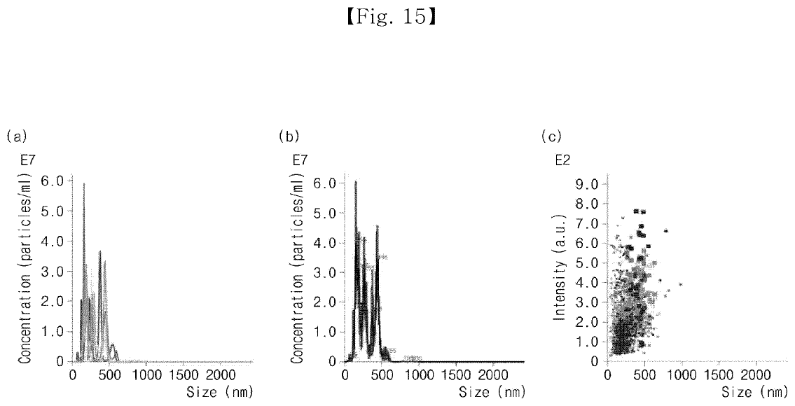

FIG. 15 is a graph for confirming size distribution of biomarkers used in the saliva pre-processing device according to the embodiment of the present invention.

Referring to FIGS. 15(a) to 15(c), the size distribution of individual biomarkers was confirmed using a NTA method. As can be seen from FIGS. 15(a) to 15(c), it was confirmed that the sizes of the separated biomarkers are distributed between about 0 to 500 nm.

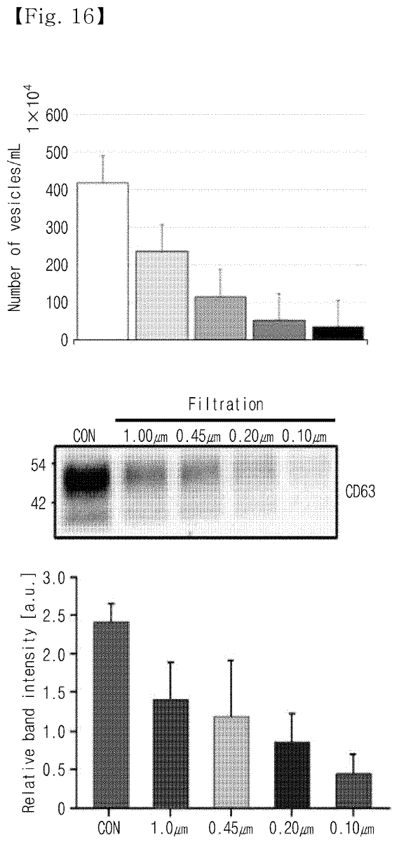

FIG. 16 is a graph showing results of filtering the biomarkers separated in FIG. 15 by the saliva pre-processing device according to the present invention.

Referring to FIG. 16, the biomarkers separated in FIG. 15 were filtered by the saliva pre-processing device, and the results thereof according to various sizes of filters were shown through comparison. As can be seen from FIG. 16, it was confirmed that the biomarkers are filtered according to the sizes of filters. Therefore, it can be seen that the biomarkers may be separated and concentrated.

Further, a first sample (sonication+3 kDa filter), a second sample (sonication+centrifuge), a third sample (3 kDa filter), and a fourth sample (centrifuge) were prepared, and then OD values, average, and protein values of each sample were shown through measurement. The measured results thereof are summarized through the following <Table 1>.

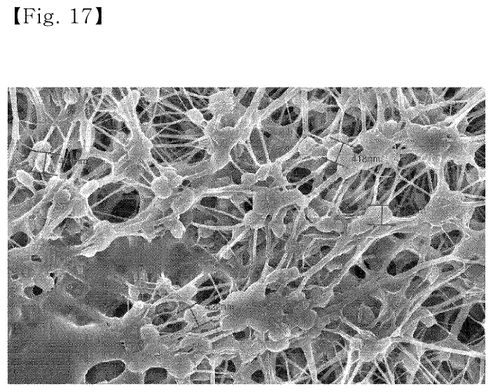

FIG. 17 is a photograph obtained by capturing a state in which the biomarkers are filtered through the saliva pre-processing device according to the embodiment of the present invention.

Referring to FIG. 17, after the biomarkers were filtered through the saliva pre-processing device according to the embodiment, the biomarkers remaining on the second filter were shown through capturing with a scanning electric microscopy (SEM). More specifically, a filter having a size of 200 nm was used as the second filter. As can be seen from FIG. 17, it was confirmed that the biomarkers having a size of about 400 nm remain on the second filter, and the biomarkers having a size of 200 nm or less are separated while passing through the filter. Therefore, it can be seen that the biomarkers may be concentrated through the second filter.

While the present invention has been described in connection with the embodiments, it is not to be limited thereto but will be defined by the appended claims. In addition, it is to be understood that those skilled in the art can substitute, change, or modify the embodiments in various forms without departing from the scope and spirit of the present invention.

### INDUSTRIAL APPLICABILITY

The device and the method for pre-processing saliva according to the embodiment of the present invention may be applied to the medical industry.

1. A saliva pre-processing device comprising:
a first filter configured to purify impurities in saliva such that the impurities in the saliva remain on a filter; and
a second filter module configured to receive the saliva from which the impurities are purified from the first filter module to concentrate a target material in the saliva such that the target material in the saliva remains on the filter,
wherein the first and second filter modules have a porous structure, and a pore size of the second filter module is smaller than a pore size of the first filter module.

2. The saliva pre-processing device of claim 1, wherein the first filter module includes:
a first filter body having an inlet and an outlet disposed to face each other; and
a first filter disposed between the inlet and the outlet in the first filter body, and
the first filter has a shape that is gradually convex from an outside toward a center thereof, and a convex portion of the first filter is disposed to face the inlet of the first filter body.

3. The saliva pre-processing device of claim 2, wherein the first filter module further includes a plurality of supporters having a rod shape extending from the outside toward the center of the first filter, and configured to support the first filter while being disposed below the first filter, and
the supporters have a slope formed in a direction from one end to the other end thereof, so that the convex portion of the first filter is formed by the supporters.

4. The saliva pre-processing device of claim 1, wherein the second filter module includes:
a second filter body having an inlet and an outlet disposed to face each other, and having a conical shape with a diameter that gradually decreases from an upper portion in which the inlet is formed to a lower portion in which the outlet is formed; and
a second filter having a rod shape extending in one direction and disposed on an inner surface of the second filter body.

5. The saliva pre-processing device of claim 4, wherein the saliva introduced from the first filter module to the second filter module moves along an inner surface of the second filter module so that the target material is concentrated by the second filter, and
the saliva in which the target material is concentrated through the second filter is discharged out of the second filter module.

6. The saliva pre-processing device of claim 4, wherein a plurality of second filters are disposed on the inner surface of the filter body, and
any one of the plurality of second filters includes a pH indicator indicating a pH of the saliva.

7. The saliva pre-processing device of claim 4, wherein the second filter is replaced depending on a type of the target material, and
a pore size of the second filter varies depending on the type of the target material.

8. The saliva pre-processing device of claim 1, wherein the first filter module and the second filter module include any one of mixed cellulose ester (MCE), polyethersulfone (PES), cellulose acetate (CA), Nylon, Teflon, polyvinylidene fluoride (PVDF), and polytetrafluoroethylene (PTFE).

9. The saliva pre-processing device of claim 1, further comprising:
a saliva cartridge coupled to the first filter module, and configured to store the saliva supplied to the first filter module; and
a saliva injection module coupled to the saliva cartridge, and configured to apply a pressure to the saliva cartridge such that the saliva is supplied from the saliva cartridge to the first filter module.

10. The saliva pre-processing device of claim 9, wherein the saliva cartridge includes a rubber packing that makes contact with the saliva injection module and forms a space for storing the saliva, and a pH indicator disposed in the space for storing the saliva, and
as the saliva injection module applies the pressure to the rubber packing, the saliva stored in the saliva storage space is supplied to the first filter module.

11. A saliva pre-processing method comprising:
preparing a saliva cartridge in which saliva is stored;
supplying the saliva stored in the saliva cartridge to a first filter having a pore of a first size;
purifying impurities in the saliva such that the impurities in a material of the saliva remain on the first filter, and passing the saliva from which the impurities are purified through the first filter;
supplying the saliva from which the impurities are purified to a second filter formed with a pore of a second size that is smaller than the first size; and
concentrating a target material in the saliva such that the target material in the saliva remains on the second filter, and passing the saliva in which the target material is concentrated through the second filter.

12. The saliva pre-processing method of claim 11, wherein the target material includes any one of protein, metabolite, and exosome.
